(19) United States
(12) Patent Application Publication
(10) Pub. No.: US 2009/0137496 A1
(43) Pub. Date: May 28, 2009

(54) Use of Active Ingredients Containing Hydroxystilbene for Preventing and/or Treating Osteoporosis

(75) Inventors: Peter Heger, Ubstadt-Weiher (DE); Reinhard Rettenberger, Goppingen (DE); Carl-Friedrich Spaich, Heiningen (DE)

Correspondence Address: EDWARDS ANGELL PALMER & DODGE LLP, P.O. BOX 55874, BOSTON, MA, 02205, US

(73) Assignee: MATSUSHITA ELECTRIC INSUTRIAL CO., LTD., OSAKA (JP)

(21) Appl. No.: 11/883,697

(22) PCT Filed: Feb. 3, 2006
(86) PCT No.: PCT/EP06/00956

§ 371 Date: Jun. 27, 2008

(30) Foreign Application Priority Data
Feb. 4, 2005 (DE) 102005005268.1
Feb. 4, 2005 (DE) 102005005271.1
Feb. 4, 2005 (DE) 102005005277.0

(51) Int. Cl. A61K 31/7034

(52) U.S. Cl. 514/25

(57) ABSTRACT

The invention relates to the use of a combination of active ingredients containing hydroxystilbene, selected from resveratrol and piceatannol precursors and functional derivatives thereof, in addition to the use of the stereoisomeric forms of said ingredients, in the form of salts or phenol respectively, for producing an agent for the prevention and/or treatment of osteoporosis.

The invention relates to the use of a combination of hydroxystilbene-containing active ingredients, selected from precursors of resveratrol and piceatannol; and the stereoisomeric forms thereof, in each case in the form of their salts or in the phenol form and functional derivatives thereof, for producing a composition for preventing and/or treating osteoporosis.

### BACKGROUND OF THE INVENTION

The term “bone remodeling” means a cyclic process of bone resorption and bone formation for the purpose of renewal or repair of old or damaged bone. The two main cell types responsible for remodeling are osteoclasts which resorb the bone, and osteoblasts which are responsible for formation of new bone.

Osteoporosis is a disease characterized by reduced bone mass. This is caused both by an increase in osteoclastic bone resorption and by a decrease in osteoblastic bone formation, resulting in greater fragility of bone and a greater risk of fracture. Because of the increasing life expectancy, osteoporosis has increasingly become a substantial health problem in industrialized countries.

An important key factor in osteoporosis is interleukin-6 (IL-6) which is induced by tumor necrosis factor α (TNF-α) (Scheidt-Nave et al, J. Clin. Endocrin. Metab. 2001; 86: 2032-2042). Both factors are multifunctional cytokines involved in proinflammatory and acute inflammatory processes. IL-6 is secreted by stromal cells and has a beneficial effect on osteoclast maturation during the first stage of osteoclastogenesis. For example, an increase in circulating IL-6 and TNF-α were observed in chronic inflammatory disorders or following natural or surgically induced menopause (Dijsselbloem et al, Endocrinology 2003; 144: 1098-1107). Thus, serum concentrations of IL-6 are very informative in relation to femoral bone loss.

It has been possible to show that an estrogen deficiency enhances the responsiveness of cells by these cytokines by upregulating the number of cytokine receptors and cofactors, and thus amplifying the effects of an increase in cytokines. In contrast thereto, it has been observed that the serum-IL-6 levels are lower in postmenopausal women receiving hormone replacement therapy (HRT) (Pfeilschifter et al, Endocrine Rev. 2002; 23: 90-119). Estrogen is thus a decisive negative regulator of IL-6, and its downregulation is impaired when estrogen levels fall.

Inhibition of IL-6 by hydroxystilbenes has been described to date only for the single substances piceatannol (Dang et al. 2004) and resveratrol (Wang et al. 2001). Use of these substances, singly or in combination, in osteoporosis because of the IL-6 inhibition is not described. Nor has there been any description of whether ERr 731® or an active ingredient combination of rhaponticin, deoxyrhaponticin, rhapontigenin and/or deoxyrhapontigenin activates estrogen receptors and thus has an osteoprotective effect.

In summary, therefore, upregulation of proinflammatory cytokines in the plasma leads to expression of further enzymes and mediators which are responsible for osteolysis.

The pharmacological interventions currently available for preventing fractures in patients with osteoporosis include one of the following two strategies:
reduction in bone resorption using bisphosphonates, calcitonin, calcium, estrogen, estrogen derivatives or selective estrogen receptor modulators;
stimulation of bone formation using fluoride salts or parathyroid hormones.

Hormone replacement therapy (HRT) using estrogen and progesterone has been employed for preventing osteoporosis in the postmenopause. HRT has been regarded as safe. However, this changed when the contrary was shown by large randomized controlled studies. There is evidence that HRT increases the risk of cardiovascular disorders in postmenopausal women (Writing Group for WHI Investigators, JAMA 2002; 288: 321-333; Chlebowski et al., JAMA 2003; 289: 3243-3253) and that it is associated with an increased risk of the occurrence of breast cancer (Million Women Study Collaborators, Lancet 2003; 362: 419-427; Schairer et al., JAMA 2000; 283: 485-491; Ross et al., J Natl Cancer Inst 2000; 92: 328-332; Colditz et al., N Engl J Med 1995; 332: 1589-1593; Magnusson et al., Int J Cancer 1999; 81: 339-334).

Based on these results, an increasing number of women are refusing to use HRT. In addition, HRT is unsuitable for the treatment of women who already had a tumor have a high risk of cancer, especially breast and endometrial cancer.

The other known therapeutic approaches are also associated with disadvantages. Thus, bisphosphonates cause side effects in the form of gastrointestinal upsets, bone and muscle pain and hypocalcemia, whereas etidronate delays the mineralization of newly formed bone tissue. Contraindications for these medicaments are renal failure, pregnancy and lactation. There is additionally the risk of interactions with calcium, iron salts and magnesium salts, thus reducing the absorption of bisphosphonates. Fluorides are contraindicated for children and young persons who are still growing and in women of child-bearing age. Calcitonin requires deep intramuscular or intravenous administration and therefore shows poor compliance and additionally leads to gastrointestinal symptoms.

For these reasons, new active ingredients are sought for preventing and treating osteoporosis, especially during the postmenopause, which are free of the side effects described above of a conventional therapy.

Since 1993, a dry extract of roots of Rheum rhaponticum has been on the market in Germany under the name Extrakt Rheum rhaponticum (ERr 731) (proprietary name Phytoestrol® N) for follicle hormone replacement therapy, for example for treating women with menopausal symptoms, juvenile oligomenorrhea and dysmenorrhea, primary and secondary amenorrhea, and endometritis. The constituents of the specific ERr 731 extract are rhaponticin, deoxyrhaponticin, rhapontigenin and deoxyrhapontigenin (table 1).

TABLE 1
Composition of the extract ERr 731

| Hydroxystilbene | Chemical name | CAS No. |
|---|---|---|
| Rhaponticin | 3,3′,5-Trihydroxy-4′- | 155-58-8 |
| | methoxystilbene 3- | |
| | O-β-D-glucopyranoside | |
| Deoxyrhaponticin | 3,5′-Dihydroxy-4′- | 30197-14-9 |
| | methoxystilbene 3-O-β- | |
| | D-glucopyranoside | |
| Rhapontigenin | 3,3′,5-Trihydroxy-4′- | 500-65-2 |
| (trans-Rhapontigenin) | methoxystilbene | |
| Deoxyrhapontigenin | 3′,5-Dihydroxy-4′- | 33626-08-3 |
| | methoxystilbene | |

All of the constituents of ERr 731 belong to the group of hydroxystilbenes. Representatives of this group have the following general formula:

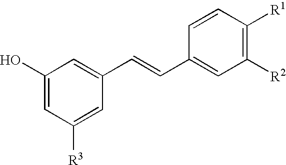

| | R¹ | R² | R³ |
|---|---|---|---|
| Resveratrol | OH | H | OH |
| Rhaponticin | OCH₃ | OH | O-Glc |
| Deoxyrhaponticin | OCH₃ | H | O-Glc |
| Rhapontigenin | OCH₃ | OH | OH |
| Deoxyrhapontigenin | OCH₃ | H | OH |
| Astringin | OH | OH | O-Glc |
| Piceatannol (astringenin) | OH | OH | OH |

Several studies have shown that the number and position of the free hydroxy and methoxy groups strongly influences the biological activity of the hydroxystilbenes (Kageura et al. Bioorganic & Medicinal Chemistry 9 (2001) 1887-1893, Matsuda et al. Biol. Pharm. Bull. 2001 (24(3) 264-267, Roberti et al. J. Med. Chem. 2003, 46, 3546-3554). The pharmacological effect of the hydroxystilbenes is moreover dependent on the presence of a glucose group (Park et al. Arch. Pharm. Res. 2002, 25(4), 528-533).

There has been only inadequate investigation of whether, and to which metabolites, the constituents of ERr 731® are degraded in the body for example after oral administration. Thus, it is merely known from investigations on the antithrombotic and antiallergic activity of rhaponticin-containing extracts from rhizoma rhei that rhaponticin is degraded by bacteria of the human intestinal tract to rhapontigenin (Park et al, Arch. Pharm. Res. 2002, 25 (4), 528-533). Metabolism of rhaponticin to piceatannol or of deoxyrhaponticin to resveratrol has not been observed to date.

JP 2000344622 relates to the provision of stabilized stilbene-containing formulations which, besides stilbene, include cyclodextrin or a derivative thereof. Rheum spp. is described as possible stilbene source. In example 3 there is a description of a cyclodextrin-containing composition for preventing osteoporosis, which includes inter alia resveratrol and a plant extract which is not characterized in detail. However, data providing evidence of the asserted effects are not provided.

US patent application 2001/0039296 describes the use of trans-resveratrol in combination with other compounds, especially phytoestrogens, for the treatment of menopausal symptoms such as osteoporosis. Preferred phytoestrogen sources are soybean derivatives, soybean isoflavones, and plants such as valerien root and kava kava. However, this document does not describe the usefulness of active ingredient combinations from Rheum rhaponticum for treating osteoporosis.

European patent EP-B 1 075 256 describes the use of monomeric or polymeric polyhydroxylated stilbenes or corresponding glycosides as antagonists of ligands of the receptors for arylhydrocarbons for treating pathologies which are induced by these harmful arylhydrocarbons. However, all the examples relate to tests with resveratrol. Also protected inter alia is the treatment of osteoporosis in women of all ages and especially in the postmenopause, and in women of any age in the event of exposure to arylhydrocarbons. Reference is made to numerous epidemiological data showing that heavy smoking and thus great exposure to arylhydrocarbons represents a risk factor for osteoporosis in men and women. However, this document does not describe the usefulness of active ingredient combinations containing no resveratrol, e.g. from Rheum rhaponticum, for treating osteoporosis, especially osteoporosis not caused by exposure to arylhydrocarbons.

US patent application 2004/0220118 describes active ingredient combinations which are said to be suitable inter alia also for the treatment of osteoporosis, these compositions including a mixture of isoflavone, an isoflavone synergist and a methylation aid. Resveratrol is described as specific example of an isoflavone synergist. However, this document does not describe the usefulness of active ingredient combinations containing no resveratrol, e.g. from Rheum rhaponticum, for treating osteoporosis.

### SUMMARY OF THE INVENTION

The present invention was therefore based on the object of finding a new way of preventing and/or treating osteoporosis.

This object has surprisingly been achieved by the use of a hydroxystilbene-containing active ingredient combination comprising at least two compounds selected from precursors of resveratrol and piceatannol; and the stereoisomeric forms thereof, in each case in the form of their salts or in the phenol form, or functional derivatives thererof, for producing a composition for preventing and/or treating osteoporosis.

The present invention is based on the identification of a novel mode of action of active ingredients and active ingredient combinations of the invention, such as, in particular, of the dry extract ERr 731®.

### DESCRIPTION OF THE FIGURES

FIG. 1 shows the result of a pharmacokinetic investigation on the ingredient rhaponticin in ERr 731® in the blood of a volunteer after oral administration of ERr 731®. Rhaponticin was detectable in the blood, but not rhapontigenin. Likewise, the metabolite thereof piceatannol was undetectable in the blood under the experimental conditions.

FIG. 2a shows the dose-dependent accumulation (AUC 0-24 h (ng×h/ml)) of rhaponticin and deoxyrhaponticin in dog plasma (M=male, F=female) after administration of ERr 731®; the aglycones rhapontigenin and deoxyrhapontigenin are undetectable; FIG. 2b shows the formation of piceatannol and resveratrol in vivo in male and female dogs 24 hours after administration of 100 mg of ERr 731®/kg of body weight.

FIG. 3 shows the effect on the IL-6 level of treatment of patients with menopausal symptoms (FA II) with ERr 731® for 15 months. It was found that the IL-6 levels were significantly reduced with ERr 731® compared with the levels before the first intake (IC).

FIG. 4 shows the change in the concentration of the bone resorption marker pyridinoline (PYD) and deoxypyridinoline (DPD) in the urine of female subjects after administration of ERr 731®.

FIG. 5 shows the reduction in IL-6 production, stimulated by administration of the cytokines IL-1β and TNFα, in the human lung carcinoma cell line A549 by the active ingredient combination ERr 731®.

FIG. 6a shows the activating effect of ERr 731® on the ERα in the osteosarcoma cell line U2OS which stably expresses ERα and thus reacts very sensitively to substances which have an affinity for this receptor. The individual substances piceatannol (FIG. 6b) and resveratrol (FIG. 6c) show no activation of the ERα in the osteosarcoma cell line U2OS, but activate relatively specifically only the ERβ.

FIG. 7 shows the experimental result on ERα activation with ERr 731®. The active ingredient combination does not activate ERα either in the Ishikawa human endometrial carcinoma line (FIG. 7a) (***=p<0.001) or in yeast cells (FIG. 7b), both of which were transfected with ERα.

### DETAILED DESCRIPTION OF THE INVENTION

### 1. Preferred Aspects of the Invention

A first aspect of the invention relates to the use of a hydroxystilbene-containing active ingredient or of a hydroxystilbene-containing active ingredient combination selected from resveratrol and piceatannol prodrugs (precursors), such as, in particular, rhaponticin, deoxyrhaponticin, rhapontigenin, deoxyrhapontigenin and astringin; and resveratrol and piceatannol; and the stereoisomeric forms thereof, especially cis and trans forms, in each case in the form of their salts or in the phenol form, or combinations of these compounds for producing a composition for the prevention and/or treatment of headache and migraines.

The invention relates in particular to the use of a hydroxystilbene-containing active ingredient combination comprising at least two compounds selected from precursors of resveratrol and piceatannol; and the stereoisomeric forms thereof, in each case in the form of their salts or in the phenol form, or functional derivatives thereof, for producing a composition for preventing and/or treating osteoporosis.

A composition produced according to the invention may in particular counteract a pathological increase in the IL-6 serum level and/or show an osteoprotective effect through a tissue-specific or bone-specific ERα activation.

A normal, i.e. not pathologically elevated, serum IL-6 level is influenced by factors such as age and gender. However, a baseline value of approximately 0 to 2 pg/ml of serum can be assumed for the IL-6 level.

Compositions of the invention are in this connection selected in particular from medicaments such as, for example, homeopathic remedies, dietary supplements, dietetic food products or other medicinal plant preparations.

Resveratrol and piceatannol prodrugs in the sense of the invention are in particular substances which can be converted, partly or completely, into resveratrol and/or piceatannol in vivo, such as, for example, in humans and/or another mammal, such as, for example, dog. Possibilities in this connection are sugar-containing (glycones, glycosides) or sugar-free (aglycones) natural or synthetic “precursors” of resveratrol or piceatannol. Typical examples of sugar-containing precursors include rhaponticin, astringin and deoxyrhaponticin. Typical examples of sugar-free precursors include rhapontigenin and deoxyrhapontigenin. The terms “prodrug” or “precursor” are, however, not to be understood as functional restriction in the context of the invention. As proven by the experimental results described hereinafter, in particular the “precursors” of the invention per se display advantageous pharmacological effects.

The active ingredients are preferably substantially present in the trans form. Salts are in particular the alkali metal and alkaline earth metal phenolates of the above compounds which have one or more free phenolic hydroxyl groups. If a plurality of hydroxyl groups is present, these can be partly or completely in the salt form.

The resulting plant extracts or individual components thereof can also be subjected to derivatization reactions in order to obtain so-called functional derivatives. These are in particular derivatives which can be converted back in the human or animal body, after administration, into the underivatized starting compound again. Mention should be made in particular of ethers and ester derivatives of the compounds used according to the invention. It is moreover possible for individual ones or all of the etherifiable or esterifiable groups in a molecule (especially the phenolic and glucosidic hydroxy groups) to be derivatized. Examples of suitable derivatives and their preparation are described for example in FR 2 835 185, which is incorporated herein by reference. Thus, mention may be made of: esters of saturated or unsaturated, aliphatic or aromatic carboxylic acids having up to 25 carbon atoms, such as 1 to 25 carbon atoms, such as, for example, saturated C₆-C₂₂ fatty acids (such as, for example, saturated unbranched fatty acids selected from caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, behenic acid); or silyl ethers, where the silicon atom carries three identical or different, straight-chain or branched, saturated or unsaturated hydrocarbon radicals having up to 20 carbon atoms, such as, for example, C₁-C₂₀ alkyl or C₂-C₂₀ alkenyl.

An active ingredient combination of at least two of the abovementioned compounds is preferably employed, such as, for example, 2, 3, 4, 5, 6, 7 or 8 individual compounds, where the group of resveratrol precursors (especially deoxyrhaponticin and deoxyrhapontigenin) and of piceatannol precursors (especially rhaponticin and rhapontigenin) is represented in each case by one compound.

In a preferred embodiment, the active ingredient or the active ingredient combination is obtainable from plants which are selected from natural plants and plants which have been modified by breeding or recombinant, genetically modified plants, which have a content of at least one of the desired ingredients which is higher by comparison with the corresponding unmodified plant. These plants are in particular selected from plant of the genus Rheum spp., Astragalus spp., Cassia spp. or Picea spp. or active ingredient-containing plant parts. Nonlimiting examples of suitable species of these genera are Rheum undulatum, Rheum palmatum, Rheum tataricum, Rheum officinale, Rheum wittrockii, Rheum altaicum, Rheum reticulatum, Astragalus complanatus, Cassia garrettiana and Picea sitchensis.

The skilled person is additionally aware that genera/species differing in provenance and differing in age (e.g. harvest at various times of the vegetation period) can be employed, in turn possibly influencing the nature, amount and composition of the active ingredients and mixtures which can be isolated therefrom. It is likewise possible in principle to use various plant parts, such as roots, rhizomes, leaves and/or stalks.

The active ingredient or the active ingredient combination is particularly advantageously obtainable from the roots, especially of Rheum rhaponticum.

In a further preferred embodiment, the active ingredient combination substantially comprises rhaponticin and deoxyrhaponticin, it being possible for the active ingredient combination substantially to comprise rhaponticin and deoxyrhaponticin in a ratio by weight of about 10:1 to 1:10, such as, for example, in a range of about 5:1 to 1:5 or 4:1 to 1:4 or 3:1 to 1:3 or 2:1 to 1:2 or about 1:1.

A further preferred active ingredient combination may comprise rhaponticin and deoxyrhaponticin, in particular in the ratios of amounts indicated above, and rhapontigenin and/or deoxyrhapontigenin. The quantitative proportion of rhapontigenin and/or deoxyrhapontigenin in the total active ingredient content may vary over a wide range and is, for example, in the range of about 0.01 to 20% by weight, in particular 0.1 to 5% by weight, based on the total active ingredient content.

Preference is further given to active ingredient combinations which have a total hydroxystilbene content, in particular a total content of deoxyrhaponticin, deoxyrhapontigenin, rhaponticin and rhapontigenin, or a total content of rhaponticin and deoxyrhaponticin, of more than 90% by weight, such as, for example, 91 to 100% by weight, or 92 to 99 or 93 to 98 or 94 to 97% by weight.

In a further preferred embodiment there is use of an active ingredient combination which is substantially free of aglycone derivatives of rhaponticin and deoxyrhaponticin, such as, in particular, resveratrol and piceatannol. “Substantially free” means an aglycone content of less than 5% by weight, in particular less than 2% by weight, such as, for example, less than 1% by weight or 0.1% by weight, such as 0 to 0.05% by weight, in each case based on the total content of rhaponticin and deoxyrhaponticin.

In a further preferred embodiment an active ingredient combination used is a plant dry extract which has a high content of glycosides, in particular glycosides of the type described above. Glycosides are in particular the above-described glycosidic precursors of resveratrol and piceatannol. These are present for example in a content of from 30 to 100% by weight, 50 to 100% by weight, but preferably in contents of more than 76% by weight, such as 76 to 99% by weight or 80 to 98% by weight or 85 to 96% by weight, in each case based on the total weight of the dry extract.

Preference is further given to active ingredient combinations which have a content of less than 0.5% by weight, such as, for example, 0-0.49% by weight or 0.001 to 0.3 or 0.01 to 0.2 or 0.01 to 0.1% by weight of anthraquinone and/or anthraquinoids (in each case based on the dry weight of the active ingredient combination). Anthraquinoids are in this connection to be understood in the widest sense as substances having a basic anthraquinone structure.

Nonlimiting examples of a suitable active ingredient combination comprising the active ingredients rhaponticin, deoxyrhaponticin, rhapontigenin, deoxyrhapontigenin, are detailed below:

60-70% by weight, such as, for example, 60-66 or 62-68% by weight, rhaponticin
30-40% by weight, such as, for example, 30-36 or 31-37% by weight, deoxyrhaponticin
0-2% by weight trans-rhapontigenin and
0-2% by weight deoxyrhapontigenin;
or
50-60% by weight, such as, for example, 53-58% by weight, rhaponticin
20-30% by weight, such as, for example, 14-28% by weight, deoxyrhaponticin
5-20% by weight, such as, for example, 10-18% by weight, trans-rhapontigenin and
0-10% by weight, such as, for example, 4-10% by weight, deoxyrhapontigenin;
in each case based on the total active ingredient content and in particular on the total content of rhaponticin, deoxyrhaponticin, rhapontigenin and deoxyrhapontigenin.

The invention also relates to the use of active ingredients or combinations thereof as defined above in combination with at least one further active ingredient which is suitable for the prevention and/or treatment of osteoporosis and differs from compounds as defined above. It is possible in particular also to combine with vitamins, minerals, further dietary supplements and/or dietetic food products.

The invention also relates to a dosage form comprising an active ingredient or an active ingredient combination as defined above in a pharmaceutically acceptable carrier.

Suitable solid dosage forms have a total active ingredient content of about 1 to 20 mg, such as, for example, 2 to 10 mg, per dose unit.

The invention relates in particular to solid dosage forms which have a sugar-free, in particular mono- or disaccharide-free, such as, for example, lactose-free, core.

Suitable solid dosage forms may be in the form of a pill, a tablet, an extrudate or granules.

Solid dosage forms in the form of a coated tablet, where appropriate with a gastro-resistant coating, are likewise suitable. Such coatings are preferably free of plasticizers such as phthalates, such as, for example, diethyl phthalate. Coating compositions suitable in particular for producing gastro-resistant, plasticizer-free coatings are selected from known natural and synthetic coating compositions (cf., for example, Voigt, Pharmazeutische Technologie, 7th edition 1993, Ullstein Mosby, Berlin). Particularly suitable coating compositions are, without being restricted thereto, shellac and cellulose derivatives such as hydroxypropylmethylcellulose derivatives such as, for example, hydroxypropylmethylcellulose acetate succinate, obtainable under the proprietary name AQOAT.

Mention should be made in particular of a solid dosage form with a total weight in the range of about 150 mg±20 mg, a core weight of 84 mg±10 mg and an active ingredient content of about 3 to 10 mg.

Further suitable solid dosage forms are those having a uniformity of active ingredient content (averaged over 10 or 20 randomly selected individual dose units) not exceeding ±5% by weight, such as, for example, ±0.1 to 4 or ±0.5 to 3 or ±1 to 2% by weight, based on the total weight of the dose unit (e.g. determined as specified in Ph. Eur. 5th edition 2005 (5.0/2.09.06.00)).

The invention further relates to a process for producing a solid dosage form where
a. the active ingredient or the active ingredient combination is mixed with the pharmaceutically acceptable carrier; and
b. the mixture is consolidated to give the active ingredient core.

For this purpose, the active ingredient or the active ingredient combination is preferably dissolved or dispersed in an inert liquid and mixed with the carrier, and the solvent is removed during or after the consolidation.

The active ingredient used according to the invention or the active ingredient combination is advantageously prepared by
a) providing an active ingredient-containing part of a medicinal plant, where appropriate in comminuted form,
b) adding an aqueous extractant thereto,
c) after the extractant has acted, obtaining a liquid extract phase from the mixture and, where appropriate, repeating the extraction several times, and
d) removing the extractant from the liquid extract phases obtained in this way.

This preferably entails carrying out an extraction with an aqueous extractant at a pH of the mixture in the alkaline range.

The extracted medicinal plant is selected in particular from plants of the genus Rheum spp, Astragalus spp, Cassia spp or Picea spp.

In a preferred variant of the preparation process, the total amount of the active ingredient or of the active ingredient combination is mixed in portions with the pharmaceutically acceptable carrier, such as, for example, Avicel or a comparable cellulose-based carrier, in particular microcrystalline cellulose, and the mixing process is repeated after each addition of carrier, but at least one or twice. In particular, a ball mill is used in this case for mixing over a period of from 30 minutes to 3 hours, such as, for example, 1 to 2 hours. It is possible to use for example conventional laboratory ball mills as described in the examples for the mixing. This results in a homogeneous and stable distribution of the active ingredient in the carrier.

In a further variant of the process, the active ingredient core is provided with a gastro-resistant, preferably plasticizer-free, coating.

In a further preferred variant in this connection, the core is sugar-coated.

The invention also relates to liquid dosage forms comprising an active ingredient or an active ingredient combination as defined above in a content of about 0.1 to 20 mg/ml, such as, for example, 0.5 to 15 or 1 to 10 or 2 to 5 mg/ml, in a solvent mixture comprising water and a pharmaceutically acceptable alcohol such as, in particular, ethanol. The solvent mixture is preferably a water/ethanol mixture with an ethanol content of from 10 to 50 or 20 to 40 or 25 to 35% by volume, such as, for example, 30% by volume. These liquid dosage forms are formulated in particular as drops for oral administration.

The invention also relates to semisolid dosage forms comprising an active ingredient or an active ingredient combination as defined above in a content of about 1 to 12, preferably 2 to 6, mg of active ingredient or active ingredient combination (per gram of the formulation) in a conventional semisolid carrier. Suitable gel-forming carriers are generally known and are selected for example from swellable cellulose derivatives such as hydroxypropylmethylcellulose, or polyacrylates such as, for example, carbopol, or gelatin. Dosage forms of this type can be used for example as vaginal gel or vaginal suppositories.

The invention also relates to a composition comprising a solid, semisolid or liquid dosage forms as defined above. Compositions in the sense of the invention are in particular pharmaceutical compositions or medicaments such as, for example, homeopathic remedies, and medicinal plant preparations.

A further aspect of the invention relates to the use of a solid, semisolid or liquid dosage form as defined above or prepared by one of the processes described above or producing a composition as defined above for the treatment of osteoporosis.

Owing to the excellent tolerability of the active ingredients or active ingredient combinations described above, the invention also relates to the use during long-term therapy, which is possible without limitation in time. The daily dose to be administered in this connection can be in the range from 0.1 to 20 mg or 0.5 to 15 mg, 1 to 10 or 4 to 8 mg of active ingredient or active ingredient combination such as, for example, ERr 731®.

The invention finally relates to the use of an active ingredient combination as defined above for reducing the IL-6 serum level and/or for activating ERα in vitro or in vivo, in particular for tissue-specific, especially bone-specific ERα activation.

### 2. Further Specific Refinements of Formulations Used According to the Invention

### 2.1 Medicaments

The invention also includes the production of pharmaceutical compositions (medicaments) for the treatment of an individual, preferably a mammal, in particular a human, productive or domestic animal. Thus, the active ingredients or active ingredient combinations described above are usually administered in the form of pharmaceutical compositions which comprise a pharmaceutically acceptable excipient with at least one active ingredient of the invention, in particular a mixture of a plurality of active ingredients of the invention, and, where appropriate, further active ingredients. These compositions can be administered for example by the oral, local, rectal, transdermal, subcutaneous, intravenous, intramuscular, intraperitoneal, intracutaneous or intranasal route.

Examples of suitable pharmaceutical formulations are solid pharmaceutical forms such as oral powders, dusting powders, granules, tablets, such as coated tablets, gastro-resistant coated tablets, dry-coated, inlay and layered tablets, pastilles, chewable tablets, suckable tablets, sachets, cachets, sugar-coated tablets, capsules such as hard and soft gelatin capsules, pessaries, suppositories or vaginal pharmaceutical forms, semisolid pharmaceutical forms such as ointments, creams, hydrogels, pastes or patches, and liquid pharmaceutical forms such as solutions, emulsions, especially oil-in-water emulsions, suspensions, for example lotions, preparations for injection and infusion, eye drops and ear drops, nose drops, nasal spray and tinctures. It is also possible to use implanted delivery devices for administering inhibitors of the invention. Liposomes, microspheres or polymer matrices can also be used in addition.

In the production of the compositions, active ingredients or active ingredient combinations of the invention are usually mixed with an excipient or diluted. Excipients may be solid, semisolid or liquid materials which serve as vehicle, carrier, adsorbent or medium for the active ingredient or the active ingredient combinations.

Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, cellulose derivatives such as, for example, methylcellulose, water, syrups and methylcellulose. The formulations may in addition comprise pharmaceutically acceptable carriers or usual ancillary substances such as lubricants, for example tallow, magnesium stearate and mineral oil; wetting agents; emulsifying and suspending agents; preservatives such as methyl and propyl hydroxybenzoates; antioxidants; antiirritants; insulating agents; tablet-coating aids; emulsion stabilizers; film formers; gel fomers; odor-masking agents; taste correctives; resins; hydrocolloids; solvents; solubilizers; neutralizers; permeation promoters; pigments; quaternary ammonium compounds; refatting and superfatting agents; ointment, cream or oil bases; silicone derivatives; spreading aids; stabilizers; sterilants; suppository bases; tablet excipients such as binders, fillers, lubricants, disintegrants or coatings; propellants; dessicants; opacifiers; thickeners; waxes; plasticizers; white oils.

An arrangement concerning this is based on expert knowledge as set forth for example in Fiedler, H. P., Lexikon der Hilfsstoffe für Pharmazie, Kosmetik und angrenzende Gebiete, 4th edition, Aulendorf: ECV-Editio-Kantor-Verlag, 1996; cf. also Hager's Handbuch der Pharmazeutischen Praxis, Springer Verlag, Heidelberg.

Solvents which are suitable according to the invention for producing formulations and which should be particularly mentioned are monohydric or polyhydric alcohols such as, in particular, ethanol, glycerol and mixtures thereof with water.

Dosage forms or pharmaceutical compositions of the invention are produced by using generally known methods of pharmaceutical technology as described for example in Voigt, Pharmazeutische Technologie, 7th edition 1993, Ullstein Mosby, Berlin.

In a preferred embodiment, a pharmaceutical composition which comprises a solid dosage form is provided. This solid dosage form in turn includes an active ingredient-containing solid core with a pharmaceutically acceptable carrier and an active ingredient content of about 1 to 20% by weight, based on the total weight of the core, where the hydroxystilbene-containing active ingredient or the hydroxystilbene-containing active ingredient combination includes a compound selected from resveratrol and piceatannol prodrugs, such as rhaponticin, deoxyrhaponticin, rhapontigenin, deoxyrhapontigenin and astringin; and resveratrol and piceatannol; and the stereoisomeric forms thereof, in each case in the form of their salts or in the phenol form, or combinations of these compounds. Preferred active ingredient combinations are as defined above.

This solid dosage form has for example a total active ingredient content of about 1 to 20 mg, such as, for example, 2 to 10 mg, per dose unit and can be in the form of a pill, a tablet, an extrudate or granules, and for example be sugar-coated. If desired, it may also have a gastro-resistant coating.

The solid dosage form is produced for example by mixing the active ingredient or the active ingredient combination with the pharmaceutically acceptable carrier, and consolidating the mixture to give the active ingredient core. This entails dissolving or dispersing the active ingredient or the active ingredient combination in an inert liquid, mixing it with the carrier and removing the solvent during or after the consolidation. The active ingredient core can then be provided where appropriate with a gastro-resistant coating before the core is sugar-coated in a conventional way.

Liquid dosage forms of the invention are produced for example by dissolving the active ingredient(s) such as, for example, an ERr731® dry extract in a suitable solvent such as, for example, a water/alcohol mixture, where appropriate together with further conventional additions. Active ingredient contents of from 0.1 to 20 or 1 to 10 mg/ml are usually adjusted in this case.

Semisolid dosage forms of the invention, such as, for example, gels, are produced for example by dissolving the active ingredient(s), such as, for example, an ERr 731® dry extract, in a suitable solvent such as, for example, a water/alcohol mixture, alcohol or glycerol, and incorporating the solution into the previously swollen gel former, where appropriate together with further conventional additions. Active ingredient contents of from 1 to 12 or 2 to 6 mg per gram of the formulation are usually adjusted in this case. Solvents which are suitable according to the invention for producing formulations and which should be particularly mentioned are monohydric or polyhydric alcohols such as, in particular, ethanol, glycerol and mixtures thereof with water, such as, for example, 10 to 50% by volume ethanol in water.

The mode and duration of administration of the medicaments of the invention are subject to the decision of the treating physician. The latter can establish a suitable dose and an appropriate dosage regimen depending on the chosen route of administration, on the efficacy of the specific active ingredient composition, the nature and severity of the disorder to be treated, the patient's condition and his response to the therapy. For example, a suitable single dose may comprise about 0.1 to 50 mg, such as, for example, 2 to 12 mg, of active ingredient or active ingredient combination as defined above, and be administered 1 to 3 times a day until the desired result of the treatment is to be observed.

### 2.2 Dietary Supplements and Food Products

The compositions of the invention also include in particular dietary supplements and food products, especially functional or dietetic food products. The food products of the invention have besides the function mainly related to nutritional value in addition a function related to active ingredients relating in particular to the active ingredient combination of the invention. They are therefore referred to as functional or dietetic food products or foodstuffs. Dietary supplements serve to supplement the daily diet with the active ingredient combination of the invention, in which case the function related to nutritional value of the dietary supplement becomes of less intrinsic importance.

The formulation base for dietary supplements and food products of the invention likewise includes physiologically acceptable ancillary substances in the widest sense, such as, for example, the abovementioned excipients. Ancillary substances in the sense according to the invention may also have a nutritional value and therefore generally be used as dietary component. Nutrients, especially essential nutrients, may also belong thereto.

Nutritional components ordinarily comprise one or more amino acids, carbohydrates or fats and are suitable for human and/or animal nutrition. They include single components, frequently vegetable, but also animal, products, especially sugars, where appropriate in the form of syrups, fruit preparations such as fruit juices, nectar, fruit pulps, purees or dried fruits, for example apple juice, grapefruit juice, orange juice, apple puree, tomato sauce, tomato juice, tomato puree, cereals products such as wheat flour, rye flour, oat flour, cornflour, barley flour, spelt flour, corn syrup, and starches from said cereals; dairy products such as milk protein, whey, yogurt, lecithin and lactose.

Examples of essential nutrients are in particular vitamins, provitamins, minerals, trace elements, amino acids and fatty acids. Essential amino acids which may be mentioned are isoleucine, leucine, lysine, methionine, phenylalanine, threonine, tryptophan and valine. These also include semiessential amino acids which must be supplied for example during growth phases or deficiency states, such as arginine, histidine, cysteine and tyrosine. Trace elements which may be mentioned are: essential trace elements and minerals such as: iron, copper, zinc, chromium, selenium, calcium, magnesium, sodium, potassium, manganese, cobalt, molybdenum, iodine, silicon, fluorine, chlorine, phosphorus, tin, nickel, vanadium, arsenic, lithium, lead, boron. Fatty acids which may be mentioned as essential for humans are: linoleic acid and linolenic acid, ARA (arachidonic acid) and DHA (docosahexaenoic acid) for infants and possibly EPA (eicosapentaenoic acid) and DHA also for adults. A comprehensive list of vitamins is to be found in “Referenzwerte für die Nährstoffzufuhr”, 1st Edition, Umschau Braus Verlag, Frankfurt am Main, 2000, edited by the Deutsche Gesellschaft für Ernährung.

Examples of suitable formulations for dietary supplementation are capsules, tablets, pills, powder sachets, liquid ampoules and bottles with dropper inserts, and the pharmaceutical forms mentioned above.

Food product formulations ordinarily have the usual form and are made available for example as breakfast preparations, in the form of mueslis or bars, sports drinks, complete meals, dietetic preparations such as diet drinks, diet meals and diet bars.

Dietary supplements and food products of the invention are produced by methods familiar to the skilled worker and requiring no further explanation (cf. for example, Hans-Dieter Belitz et al. Lehrbuch der Lebensmittelchemie. Springer-Lehrbuch 5th revised edition 2001. XLIV, 1059 Verlag: SPRINGER, BERLIN)

The content of active ingredients/active ingredient combinations of the invention in the above dietary supplements and food products can vary over a wide range and is for example in a range from 0.01 to 10% by weight, such as, for example, 0.1 to 1% by weight.

The invention is now further explained by means of the following nonlimiting examples and with reference to the appended figures.

2.3 Preparation of a Drug Extract which can be Used According to the Invention

Drug extracts which can be used according to the invention are preferably prepared by
a) providing a hydroxystilbene-containing part of a medicinal plant, where appropriate in comminuted form,
b) adding an aqueous, organic or aqueous-organic extractant thereto,
c) after the extractant has acted, obtaining a liquid extract phase from the mixture, and where appropriate repeating the extraction several times, and
d) removing the extractant from the liquid extract phases obtained in this way.

In particular, the extract obtained in this way includes at least one compound selected from rhaponticin, deoxyrhaponticin, rhapontigenin, deoxyrhapontigenin as salt or in phenolic form, in a stereoisomeric form thereof, such as cis or trans form, or as mixture of such stereoisomeric forms.

However, the extracted hydroxystilbenes are preferably substantially in the trans form. Salts are in particular the alkali metal and alkaline earth metal phenolates of the above compounds which have one or more free phenolic hydroxyl groups. If a plurality of hydroxyl groups is present, they may be partly or completely in the salt form.

The resulting plant extracts or individual components thereof can, as already mentioned, also be subjected to derivatization reactions in order to obtain so-called functional derivatives.

An active ingredient combination of at least two of the abovementioned compounds is preferably obtained, such as, for example, 2, 3, 4, 5, 6, 7 or 8 Individual compounds, with the group of resveratrol precursors (especially deoxyrhaponticin and deoxyrhapontigenin) and of piceatannol precursors (especially rhaponticin and rhapontigenin) each being represented by one compound.

A further preferred embodiment of the process of the invention provides an extract which has a high content of glycosides, in particular glycosides of the type described above, such as, for example, a content of from 30 to 100% by weight, 50 to 100% by weight, 60 to 99% by weight or 80 to 98% by weight or 85 to 96% by weight, in each case based on the total weight of the resulting dry extract. A “dry extract” in the sense of the invention is present in particular when the residual moisture content of water and/or organic liquid (such as extractant) is less than about 5% by weight, in particular less than 2% by weight, such as, for example, 0 to 1.5% by weight or 0.1 to 0.5% by weight, in each case based on the total weight of the resulting dry extract.

A further preferred embodiment provides an extract which is substantially free of aglycone derivatives of rhaponticin and deoxyrhaponticin, such as, in particular, resveratrol and piceatannol. “Substantially free” means an aglycone content of less than 5% by weight, in particular less than 2% by weight such as, for example, less than 1% by weight or 0.1% by weight, such as 0 to 0.05% by weight, in each case based on the total weight of rhaponticin and deoxyrhaponticin.

Active ingredient combinations which are further preferably prepared are those having a total hydroxystilbene content of more than 90% by weight such as, for example, 91 to 100% by weight, or 92 to 99 or 93 to 98 or 94 to 97% by weight.

Further active ingredient combinations which are preferably prepared are those having a content of less than 0.5% by weight, such as, for example, 0-0.49% by weight or 0.001 to 0.3 or 0.01 to 0.2 or 0.01 to 0.1% by weight, of anthraquinone and/or anthraquinoids (in each case based on the dry weight of the active ingredient combination). Anthraquinoids are in this case to be understood in the widest sense as substances having a basic anthraquinone structure.

In a preferred embodiment, the medicinal plant to be extracted is selected from natural plants and plants modified by breeding or recombinant, genetically modified plants which have a content of at least one of the desired ingredients which is higher by comparison with the corresponding unmodified plant. These plants are selected in particular from plants of the genus Rheum spp., Astragalus spp., Cassia spp. or Picea spp. or active ingredient-containing plant parts. Nonlimiting examples of suitable species of these genera are Rheum undulatum, Rheum palmatum, Rheum tataricum, Rheum officinale, Rheum wittrockii, Rheum altaicum, Rheum reticulatum, Astragalus complanatus, Cassia garrettiana and Picea sitchensis. It is additionally preferred to employ medicinal plants as single varieties.

The skilled worker is additionally aware that genera/species differing in provenance and differing in age (i.e. harvest at various times of the vegetation period) can be employed, in turn possibly influencing the nature, amount and composition of the hydroxystilbenes and mixtures which can be isolated therefrom. It is likewise possible in principle to use various plant parts such as roots, rhizomes, leaves and/or stalks.

The respective plant part or mixture of plant parts can, if expedient, be mechanically treated such as, for example, ground, chopped, reeled, crushed or cut. If expedient, predrying is also possible, such as, for example, 2 hours to 2 days at 30 to 50° C., in order to reduce the liquid content.

The hydroxystilbene-containing part of the medicinal plant used for the extraction is in particular the root of the medicinal plant, such as, for example, of Rheum rhaponticum.

The invention relates in particular to a process in which a hydroxystilbene-containing percolate is prepared from the drug. A “percolation” means a continuous extraction of soluble substances from a drug by continual renewal of the solvent. This results in a permanent concentration gradient, so that a large part of all the soluble substances goes into the extract.

An alternative possibility is also a continuous or periodic mixing of the batch such as, for example, by stirring or shaking.

The temperature during the extraction according to the invention is usually in the range from 10 to 50° C., such as, for example, 25 to 35° C. The pressure is usually atmospheric pressure. If a speeding up of the rate of extraction or quality of the extract can be achieved, the pressure may also be varied during the extraction, such as, for example, raised or lowered.

The extraction may take, depending on the chosen conditions such as the nature of the drug, batch size, extractant and temperature used, from 1 hour to several days, such as, for example, 10 to 72 hours.

The extraction process can if necessary be repeated several times in order to ensure that isolation in particular of the desired ingredients is as complete as possible. The ratio by weight of introduced drug to liquid extractant may vary over a wide range and from extraction step to extraction step. The ratio by weight of drug to extractant is typically in the range from 10:1 to about 1:200 or about 1:2 to 1:50, or 1:4 to 1:10.

In one variant of the process, an extraction is carried out with an aqueous extractant which is substantially free of organic solvent, such as, in particular, water, preferably purified water, at a pH of the mixture in the alkaline range, with the pH of the mixture being in particular in the range from about 11 to 12, such as, for example, about 11.3 to 11.8.

The pH of the mixture is adjusted for example with the aid of an inorganic base selected from alkali metal and alkaline earth metal hydroxides such as, for example, calcium hydroxide or calcium oxide. It is possible for this purpose for example to prepare a concentrated quicklime solution by dissolving 3 to 8 parts of CaO in 20 parts of purified water. This solution is strongly alkaline and has a pH in the range from about 12 to 13, such as, for example, of about 12.4 to 12.6.

The ratio of the amounts of introduced drug to base such as, for example, calcium hydroxide (calculated as calcium oxide) can be according to the invention in the range from about 5:1 to 20:1, such as about 8:1 to 12:1 or 9:1 to 11:1.

The process is preferably carried out in such a way that the desired hydroxystilbenes are precipitated from the resulting alkaline liquid extract phase, for example by adjusting the pH of the extract to a value in the range from about 3 to 4, such as, for example, 3.2 to 3.8, or 3.4-3.6, and, where appropriate, subsequently removing the precipitate, washing where appropriate and drying where appropriate.

Used for the acidification is any inorganic or organic acid, such as, for example, hydrochloric acid or sulfuric acid, but in particular organic acids such as formic acid or acetic acid.

Before removal of the precipitate it may be expedient to leave the batch to stir for some hours or days in order to achieve precipitation which is as quantitative as possible of the desired extracted ingredients.

The precipitate can be washed for example with purified water, and this serves in particular to remove remaining acid.

Remaining liquid is removed from the extract by drying, e.g. at 30 to 50° C. or 35 to 45° C., for example over a period of from 1 to 100 hours, until the residual moisture is in the range indicated above. The drying takes place in a manner known per se, e.g. in a drying oven. Freeze drying is likewise possible.

### EXPERIMENTAL SECTION

### General Methods:

Determination of Stilbenes by High-Pressure Liquid Chromatography (HPLC) in the Dry Extract from Rhapontic Rhubarb Root

### a) Sample Preparation:

50 mg of extract, mixed with 40 ml of a mixture of acetone and water (1:1) in an amber glass vessel, treated in an ultrasonic bath for 15 minutes and made up to 50 ml with the solvent mixture and then diluted 1:10 with the solvent mixture.

### b) Procedure for the Chromatography:

A high-pressure liquid chromatography (HPLC) is carried out on a portion of the solution obtained above, with the following system parameters:

| Sample loop: | 20 μl | |
|---|---|---|
| Column: | Lichrospher 5 μ RP 18, 250 × 4 mm | |
| Precolumn: | Lichrospher 5 μ RP 18, 5 × 4 mm | |
| Column temperature: | 25° C. | |
| Eluent A: | Acetonitrile/dist. water/phosphoric acid 85%, | |
| | 15/85/0.05 (parts by volume) | |
| Eluent B: | Acetonitrile/dist. water/phosphoric acid 85%, | |
| | 80/20/0.05 (parts by volume) | |
| Flow rate: | 1.5 ml/min | |
| Column flushing: | 15 min with eluent 50% B; equilibration time | |
| | 15 min | |
| Detection: | 310 nm | |
| HPLC: | Kontron Kroma 2000 | |
| Gradient: | Time | % B |
| | 0.0 | 0 |
| | 0.5 | 0 |
| | 7.5 | 75 |
| | 8.5 | 100 |
| | 9.5 | 0 |
| | 12.5 | 0 |

The retention times resulting under the system conditions indicated above are as follows:

| | |
|---|---|
| Rhaponticin: | about 5.5 min |
| Deoxyrhaponticin: | about 6.8 min |
| Rhapontigenin: | about 7.2 min |
| Deoxyrhapontigenin: | about 9.0 min |

For a quantitative determination, the respective peak areas are found and compared with the corresponding peak areas of a standard extract of known composition.

### Preparation Example 1

### Preparation of the Dry Extract ERr 731 from Rhapontic Rhubarb Root with an Aqueous Calcium Hydroxide Solution

A dry extract is prepared from rhapontic rhubarb root employing the following:

| | |
|---|---|
| Drug (radix rheum rhaponticum) | 50.0 kg |
| Calcium oxide | 5.0 kg |
| Purified water | 190.0 kg |
| Acetic acid (as necessary to adjust the require pH) | |

The yield which can be achieved in this case is between 2 and 3 kg per 50 kg of drug.

The preparation takes place in the following steps:

a) Firstly 5 kg of calcium oxide are introduced into a plastic tub and made into a slurry with 20 kg of purified water. The formation of calcium hydroxide (quicklime) which takes place under these conditions leads to a large rise in temperature of the solution. The calcium hydroxide can therefore be used further only after cooling. The temperature of the solution is then 30° C. to 35° C.
b) 50 kg of drug are introduced into a mixer, and the abovementioned quicklime is added. In order to remove the quicklime as completely as possible from the plastic tub, it is rinsed with 10 kg of purified water. This washing liquid is likewise put in the mixer.
c) The drug homogeneously mixed with quicklime is introduced into a percolator and covered with 160 kg of purified water. The percolator remains closed for 48 hours. The percolate is then collected in a suitable vessel at a flow rate of 50 ml/min. The percolation is continued until no further percolate emerges. The drug mass is not squeezed out after completion, but is discarded.
d) While monitoring continuously, concentrated acetic acid is added to the percolate until a pH in the range from 3.4 to 3.6 is reached. In order to achieve precipitation of the extract which is as complete as possible, the mixture is left to stand for 5 days.
e) The dry extract is obtained by filtration through Büchner funnels under applied vacuum. Finally, the extract is washed with 10 to 20 kg of purified water.
f) The dry extract obtained after filtration is dried in a drying oven at 40° C. until a residual moisture tolerance not exceeding 1% is reached.

Rhaponticin is readily soluble in aqueous solutions with an alkaline pH range, whereas it is precipitated as yellowish substance in the acidic pH range (pH 3.4-3.6). Use is made of this for its isolation. Since, besides other organic acids, the root in particular has a high content of oxalic acid (⅔ in water-soluble and ⅓ in bound form), this must be neutralized during the isolation in order to prevent the pH drifting into the acidic range and thus to inhibit premature precipitation of the rhaponticin. This is achieved by using calcium oxide. The latter is employed as quicklime solution with a pH of 12.4-12.6.

Homogeneous mixing of the quicklime with the drug alters the pH of the mixture. It is then in the range from 11.3 to 11.8, thus preventing precipitation of rhaponticin, because the phenolic form has been converted into a phenolate form. Despite the high oxalic acid content, the pH can be kept in the alkaline range. This is attributable to the fact that the calcium hydroxide reacts with oxalic acid and forms insoluble and nontoxic calcium oxalate.

Rhaponticin is extracted from the root by the subsequent percolation with purified water. After completion of the percolation, a pH of 3.4 to 3.6 is adjusted by adding acetic acid. This pH shift from the alkaline to the acidic range leads to a precipitation of rhaponticin through conversion back into the phenolic form. In order to achieve precipitation of rhaponticin which is as complete as possible, the mixture is left to stand for 5 days. It is then filtered. Rhaponticin remains as yellowish substance on the filter.

The above statements about rhaponticin apply correspondingly to the other hydroxystilbene active ingredients isolated according to the invention.

### Preparation Example 2

### Preparation of a Dry Extract from Rhapontic Rhubarb Root with Various Organic Solvents

The constituents mainly detectable in the rhapontic rhubarb root used as drug here belong to the group of hydroxystilbenes. Present from this group in the roots are rhaponticin (Rh) with a content of about 6% and deoxyrhaponticin (DRh) with a content of about 4%.

It is possible by exposure to the solvent systems indicated below, in a 100-fold quantity at room temperature for 10 minutes with shaking or stirring, to extract the proportions summarized below:

| | | |
|---|---|---|
| Ethanol 86% | Rh | 100.8% |
| | DRh | 99.5% |
| Ethanol 15% | Rh | 77.1% |
| | DRh | 75.5% |
| Acetone | Rh | 88.3% |
| | DRh | 96.6% |
| Water, alkaline | Rh | 75.5% |
| (pH 11, adjusted with CaO solution) | DRh | 60.5% |

No useful results were achieved with heptane.

The respective yields of crude extracts in proportions by mass (based on drug employed) are as follows:

| | |
|---|---|
| Ethanol 86% | 35.5% |
| Ethanol 15% | 32.2% |
| Acetone | 21.4% |
| Heptane | 0% |
| Water, alkaline | 4.5% |

Extraction of rhapontic rhubarb root with ethanol-water mixtures leads to an extract which, besides the main constituents rhaponticin (about 30%) and deoxyrhaponticin (about 22%), comprises a further stilbene, which has not as yet been investigated, in a proportion of about 20% in the extract. Besides these, the aglycones rhapontigenin (about 8%) and deoxyrhapontigenin (about 2%) and a further 9 compounds which total about 20% are obtained.

The results on extraction with acetone are fundamentally the same.

Extraction with alkaline water (cf. conditions in preparation example 1) leads to an extract of greater purity.

The main constituents rhaponticin and deoxyrhaponticin are present in a proportion of about 97% in the dry extract. Rhapontigenin and deoxyrhapontigenin together amount to a proportion of 1.1% of the extract, whereas the stilbene which has not yet been investigated is present in a proportion of only 0.2%. A further 3 compounds are present in a proportion of 2.5%.

### Formulation Example 1

### Production of a Solid Dosage Form—Minitablet

### 1. Production of the Tablet Core:

A solid tablet core is produced using the following active ingredients and ancillary substances in the stated ratios of amounts (P=parts by weight). The ingredients are mixed and tableted in three different ways:

### a) Tablet Core Formulation:

| Purified dry extract according to | | |
|---|---|---|
| preparation example 1 | | |
| from rhapontic rhubarb root (ERr 731 ®) | 3.6 | P |
| Microcrystalline cellulose (e.g. Avicel ®) | 57.0 | P (±40%) |
| Sorbitol | 8.0 | P ″ |
| Talc | 2.5 | P ″ |
| Makrogol 6000 (polyglycol) | 1.6 | P ″ |
| Polyvidone (K value about 25, e.g. Kollidon ® 25) | 1.6 | P ″ |
| Sodium dodecyl sulfate (e.g. Texapon ® K 12) | 0.5 | P ″ |
| Magnesium stearate (vegetable) | 0.8 | P ″ |
| | 75.6 | P (±40%) |

It is possible by varying the weighed amount of ERr 731® and/or varying the amount of microcrystalline cellulose to obtain any desired ERr 731® contents in the untreated core (such as, for example, 2, 4, 6, 8, 10, 12 mg per tablet).

### b) Mixing of Drug and Carrier

Mixing Variant a:

1.2 P of ERr® 731 are triturated in portions with Avicel® in a ball mill and then, after addition of the other ancillary substances, mixed and tableted as described below.
Mixing Variant b:

ERr 731 (1 g/l of solvent) is dissolved in a suitable solvent (e.g. ethanol/water mixture 86% v/v ethanol) and adsorbed on Avicel®, dried (at 40° C. for at least 48 hours) and, after addition of the other ancillary substances, mixed and tableted as described below.
Mixing Variant c:

The total amount of Avicel® is divided into three equal portions. The first portion is mixed with the total amount of ERr731® and triturated in a laboratory ball mill (e.g. type 1-25 LK, Alpine, Augsburg) for at least 120 minutes. The second portion of Avicel® is then added, and the mixture is again triturated in the laboratory ball mill for at least 120 minutes. After addition of the third portion of Avicel®, brief mixing is again carried out. Subsequently, after addition of the other ancillary substances, mixing and tabletting are carried out as described below.

It is surprisingly possible with this mixing variant to reduce markedly the tendency to inhomogeneity and, even with small dose units, to adjust an extremely uniform active ingredient content of not more than ±5% by weight (determined according to Ph. Eur. 5th edition 2005 (5.0/2.09.06.00)).

### c) Tableting:

The mixture of Avicel® and active ingredient is sieved through a sieving machine (sieve diameter 1.2 mm) into a suitable mixing container and, after addition of the stated tabletting aids (without magnesium stearate), mixed in a suitable mixer (e.g. drum hoop mixer of type Standard RR M 200, from Engelsmann AG/Ludwigshafen) for at least 30 min. Addition of magnesium stearate is followed by mixing again for at least 5 min.

The compression takes place in a suitable tablet press (e.g. rotary of type Perfecta Fette 2000, from Fette/Schwarzenbeck):

| | |
|---|---|
| Core weight: | 84 mg ± 4.2 mg maximum variation |
| Punch: | 7 mm diameter, domed |

The ERr-731 content per core is about 4 mg±5%.

### 2. Production of the Gastro-Resistant Coated Tablet

After removal of dust from the tablet cores with Eudragit, a gastro-resistant coating of cellulose acetate phthalate and diethyl phthalate, dissolved in isopropanol and ethyl acetate, is applied to the tablet cores using a coating system.

Macrogol is dissolved in purified water. The ingredients sugar (sucrose or isomalt), calcium carbonate, talc, titanium dioxide and the two povidones are mixed and stirred into the liquid. The suspension is stirred in a jet flow mixer (e.g. Rototron of type RTA 70-50) for 20 minutes.

The sugar-coating suspension is applied to the sealed core with the aid of an automatic coater. The process is repeated until an average weight of 150 mg per coated core is reached. Finally, the polishing wax is applied and then rolling is continued until a high gloss is obtained.

### Final Weight of the Gastro-Resistant Coated Tablet:

150 mg±7.5 mg maximum variation.

In this way, two different tablet forms—one containing sugar and one sugar-free—are produced, employing the respective ancillary substances in the parts by weight indicated below.

a) Gastro-Resistant Coated Minitablet—Containing Sugar—with Plasticizer in the Coating

Ancillary substance:

| | | | |
|---|---|---|---|
| Coating: | Eudragit L12.5% dry matter | 1.350 | kg (±40%) |
| | Diethyl phthalate | 1.749 | kg ″ |
| | Cellulose acetate phthalate | 7.770 | kg ″ |
| | Isopropyl alcohol | 75.600 | kg ″ |
| | Ethyl acetate | 77.600 | kg ″ |
| | Talc | 2.000 | kg ″ |
| Sugar coating: | Talc | 7.182 | kg ″ |
| | Sugar | 28.747 | kg ″ |
| | Calcium carbonate | 6.410 | kg ″ |
| | Titanium dioxide E 171 | 0.635 | kg ″ |
| | Povidone | 0.756 | kg ″ |
| | (K value about 25, e.g. | | |
| | Kollidon ® 25) | | |
| | Povidone (K value about 90) | 0.332 | kg ″ |
| | Macrogol 35,000 | 0.635 | kg ″ |
| | Water | 10.500 | kg ″ |
| Polish: | 95% carnauba wax, | 0.108 | kg ″ |
| | 5% bleached wax | | |
| | (e.g. Capol 1295 PH) | | |

b) Gastro-Resistant Coated Minitablet—Sugar-Free—with Plasticizer in the Coating

Ancillary substance:

| | | | |
|---|---|---|---|
| Coating: | Eudragit L12.5% dry matter | 1,350 | kg (±40%) |
| | Diethyl phthalate | 1.749 | kg ″ |
| | Cellulose acetate phthalate | 7.770 | kg ″ |
| | Isopropyl alcohol | 75.600 | kg ″ |
| | Ethyl acetate | 77.600 | kg ″ |
| Sugar coating: | Talc | 7.482 | kg ″ |
| | Sorbitol and/or isomalt | 28.747 | kg ″ |
| | Calcium carbonate | 6.410 | kg ″ |
| | Titanium dioxide E 171 | 0.635 | kg ″ |
| | Povidone | 0.756 | kg ″ |
| | (K value about 25, e.g. | | |
| | Kollidon ® 25) | | |
| | Povidone (K value about 90) | 0.332 | kg ″ |
| | Macrogol 35,000 | 0.635 | kg ″ |
| | Water | 10.500 | kg ″ |
| Polish: | 95% carnauba wax, | 0.108 | kg ″ |
| | 5% bleached wax | | |
| | (e.g. Capol 1295 PH) | | |

### Formulation Example 2

### Production of a Solid Dosage Form—Minitablet Containing Sugar without Plasticizer

### 1. Production of the Tablet Core

Production takes place in analogy to formulation example 1.

### 2. Production of the Gastro-Resistant Coated Tablet

Production takes place in analogy to formulation example 1, but with use of shellac (variant A) or Aqoat (variant B) instead of cellulose acetate phthalate/diethyl phthalate (plasticizer).

### a) Variant A

| Ancillary substances: | | kg (±40%) |
|---|---|---|
| Coating: | Eudragit L12.5% dry matter | 0.400 |
| | CAPOL 5270 PH 8% | 60.000 |
| | (shellac solution) = | |
| | 4.8 kg dry matter (shellac) | |
| | Isopropyl alcohol | 4.000 |
| | Ethanol 96% | 3.200 |
| | Talc | ..2.000 |
| Sugar coating: | Talc | 7.182 |
| | Sugar | 28.747 |
| | Calcium carbonate | 6.410 |
| | Titanium dioxide E 171 | 0.635 |
| | Polyvidone | 0.756 |
| | (K value about 25, e.g. | |
| | Kollidon ® 25) | |
| | Povidone (K value: about 90) | 0.332 |
| | Macrogol 35,000 | 0.635 |
| | Water | 10.500 |
| Polish: | 95% carnauba wax | 0.108 |
| | 5% bleached wax | |
| | (e.g. Capol 1295 PH) | |

### b) Variant B

| Ancillary substances: | | kg (±40%) |
|---|---|---|
| Coating: | Eudragit L12.5% dry matter | 0.400 |
| | Aqoat | |
| | Hydroxypropylmethylcellulose | 5.420 |
| | acetate succinate | |
| | Distilled water | 12.500 |
| | Isopropyl alcohol | 4.000 |
| | Ethanol 86% | 55.000 |
| Sugar coating: | Talc | 9.182 |
| | Sugar | 28.747 |
| | Calcium carbonate | 6.410 |
| | Titanium oxide E 171 | 0.635 |
| | Polyvidone | 0.756 |
| | (K value about 25, e.g. | |
| | Kollidon ® 25) | |
| | Povidone (K value: about 90) | 0.332 |
| | Macrogol 35,000 | 0.635 |
| | Water | 10.500 |
| Polish: | 95% carnauba wax | 0.108 |
| | 5% bleached wax | |
| | (e.g. Capol 1295 PH) | |

### Formulation Example 3

### Production of a Solid Dosage Form—Minitablet Sugar—Free without Plasticizer

### 1. Production of the Tablet Core

Production takes place in analogy to formulation example 1, but using isomalt instead of Avicel.

### 2. Production of the Gastro-Resistant Coated Tablet

Production takes place in analogy to formulation example 2, but using isomalt instead of sugar.

### a) Variant A

| Ancillary substances: | | kg (±40%) |
|---|---|---|
| Coating: | Eudragit L12.5% dry matter | 0.400 |
| | CAPOL 5270 PH 8% | 60.000 |
| | (shellac solution) = | |
| | 4.8 kg dry matter (shellac) | |
| | Isopropyl alcohol | 4.000 |
| | Ethanol 96% | 3.200 |
| | Talc | 2.000 |
| Sugar coating: | Talc | 7.182 |
| | Isomalt | 28.747 |
| | Calcium carbonate | 6.410 |
| | Titanium oxide E 171 | 0.635 |
| | Polyvidone | 0.756 |
| | (K value about 25, e.g. | |
| | Kollidon ® 25) | |
| | Povidone (K value: about 90) | 0.332 |
| | Macrogol 35,000 | 0.635 |
| | Water | 10.500 |
| Polish: | 95% carnauba wax | 0.108 |
| | 5% bleached wax | |
| | (e.g. Capol 1295 PH) | |

### b) Variant B

| Ancillary substances: | | kg (±40%) |
|---|---|---|
| Coating: | Eudragit L12.5% dry matter | 0.400 |
| | Aqoat | 5.420 |
| | Distilled water | 12.500 |
| | Isopropyl alcohol | 4.000 |
| | Ethanol 86% | 55.000 |
| | Talc | ..2.000 |
| Sugar coating: | Talc | 7.182 |
| | Isomalt | 28.747 |
| | Calcium carbonate | 6.410 |
| | Titanium oxide E 171 | 0.635 |
| | Polyvidone | 0.756 |
| | (K value about 25, e.g. | |
| | Kollidon ® 25) | |
| | Povidone (K value: about 90) | 0.332 |
| | Macrogol 35,000 | 0.635 |
| | Water | 10.500 |
| Polish: | 95% carnauba wax | 0.108 |
| | 5% bleached wax | |
| | (e.g. Capol 1295 PH) | |

### Formulation Example 4

### Production of a Semisolid Dosage Form—Vaginal Gel

Production takes place using conventional methods by the two following variants:

### a) Variant A:

Hydroxypropylmethylcellulose (hypromellose USP) or another gel former is allowed to swell with 2-10% by weight in purified water. The ERr 731® (preparation example 1), dissolved in glycerol, is then incorporated. The amount of glycerol may be up to 50% of the weight of the gel. ERr 731® can be dissolved up to 0.5% by weight in glycerol. If necessary, preservatives (e.g. sorbic acid and its salts) can be added to the gel. Adjustment of the pH is also possible. As alternative to glycerol it is also possible to use 30-86% by volume ethanol.

### b) Variant B:

Carbomer (Carbopol) is dissolved with 0.5-5% by weight in purified water, and the desired pH is adjusted (e.g. KOH, NaOH, NH₃). If necessary, a preservative (e.g. sorbic acid and its salts) is admixed. After formation of a clear gel, ERr 731® (preparation example 1) is dissolved up to 0.5% by weight in 30-86% by volume ethanol and added. As alternative to ethanol, it is also possible to use glycerol.

### Formulation Example 5

### Production of a Semisolid Dosage Form—Vaginal Suppositories

Suppositories with a size of 1 to 15 g with a content of 1 to 12 mg of ERr 731® (preparation example 1) dissolved in glycerol (85% n 20/D=1.45085) are produced in a conventional way by two different variants.

### a) Variant A:

| Formulation: | |
|---|---|
| Gelatin | 1 part |
| Purified water | 2 parts |
| Glycerol 85% (+ERr 731 ®) | 5 parts |

### b) Variant B:

Same formulation but with suitable preservatives such as, for example, sorbate, benzoate, PHB ester.

The gelatin is introduced in each case into purified water and allowed to swell until the mixture has become glassy. Glycerol 85% with active ingredient is then added and heated, but not above 65° C. The suppositories are then cast in a conventional way.

### Formulation Example 6

### Production of a Liquid Dosage Form—Drops

a) Dissolving tests with ERr 731 ® in ethanol and glycerol:

| Content of the extract: | |
|---|---|
| 61.9% rhaponticin | |
| 29.9% deoxyrphaponticin | |
| Test A: 200 mg of dry extract in 50 ml of glycerol R: | |
| 55.1% rhaponticin | (89.0% of theory) |
| 27.1% deoxyrhaponticin | (90.6% of theory) |
| Test B: 200 mg of dry extract in 50 ml of ethanol 30% R: | |
| 52.2% rhaponticin | (84.3% of theory) |
| 25.2% deoxyrhaponticin | (84.2% of theory) |
| Test C: 200 mg of dry extract in 50 ml of ethanol 50% R: | |
| 58.8% rhaponticin | (95.0% of theory) |
| 29.0% deoxyrhaponticin | (97.0% of theory) |
| Test D: 200 mg of dry extract in 50 ml of ethanol 86% R: | |
| 59.8% rhaponticin | (96.6% of theory) |
| 29.5% deoxyrhaponticin | (98.7% of theory) |

### b) Production of Drops:

Drops are produced by dissolving dry extract according to test B in ethanol 30% R and filtering where appropriate.

### Test Example 1

### Pharmacokinetics and In Vivo Accumulation and Metabolism of the Ingredients of ERr 731®

### a) Pharmacokinetics of ERr 731® Ingredients in Female Subjects

The intention was to check whether, after oral intake of ERr 731®, one of the ingredients of this active ingredient combination can be found again in the blood, in order to demonstrate that at least one of the constituents of this active ingredient combination or its metabolites is bioavailable.

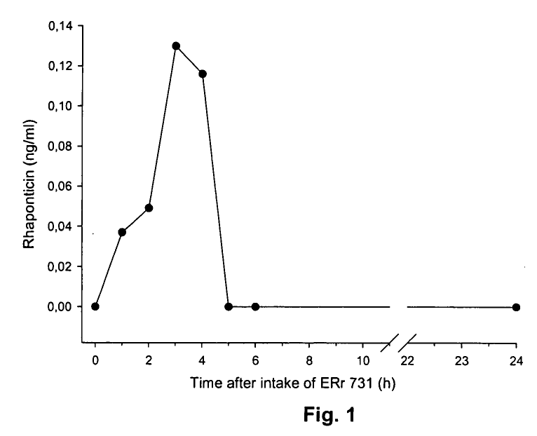

A volunteer took 10 tablets of ERr 731® (dosage=40 mg of ERr 731®) with liquid in the morning (8.00 h). Subsequently, 10 ml of blood was taken at various times (as indicated in FIG. 1) and the plasma was obtained by centrifugation.

These plasma samples were processed as follows: 500 μl of plasma were mixed with 25 μl of an internal standard working solution (2.5 ng/μl rhaponticin or rhapontigenin in methanol) and then mixed with 500 μl of isotonic NaCl solution and 2.5 ml of diethyl ether/butanol (9/1; v/v). After shaking and centrifugation (10 minutes at 4600 rpm), about 2 ml of the supernatant were removed and dried under a stream of nitrogen (at 60° C.). The pellet was taken up in 50 μl of methanol. Addition of 200 μl of distilled water was followed by renewed mixing, and 200 μl were pipetted into autosampler tubes (light-protected). 30 μl of the samples were injected for analysis into an LC-MS/MS system (PE Sciex API 3000). Chromatographic separation of the analytes took place on a Phenomenex Polymer X column with a gradient of an ammonium buffer solution and an acetonitrile/methanol mixture as mobile phase.

The analyzed results are summarized in FIG. 1. Rhaponticin was detected in the blood, with a maximum at 3-4 hours (FIG. 1), whereas rhapontigenin could not be found. Since rhaponticin is one of the main ingredients of ERr 731®, it can be assumed that rhaponticin is an activity-codetermining ingredient of ERr 731®, and is thus partly responsible for the antiosteoporotic activity of the complete extract. This is all the more surprising since it was previously assumed that only the aglycones, but not the glycosylated hydroxystilbenes, are active (Park et al., Arch Pharm Res. 2002; 25:528-533).

### b) Vivo Accumulation and Metabolism of the Ingredients of Err 731® in Dog Plasma

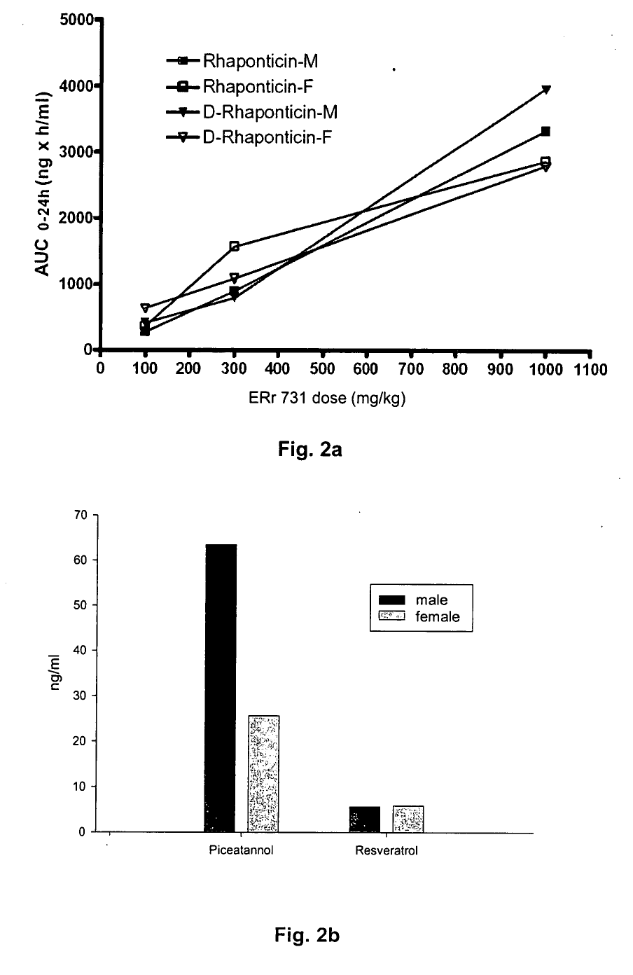

1) 20 male and 20 female dogs (pure-bred beagles, weight 6-9 kg, age 6-8 months) received 100 (4 animals each), 300 (4 animals each) and 1000 (6 animals each) mg/kg of body weight/day ERr 731®. On day 1, 5 ml of blood were taken from the animals after 0, 0.5, 1, 2, 4, 8 and 24 hours in each case, and plasma was obtained. The analysis as described in section a) was carried out thereon in order to detect rhaponticin, deoxyrhaponticin, rhapontigenin, deoxyrhapontigenin, resveratrol and piceatannol in blood. The results of the test are depicted in FIG. 2a.

2) For further elucidation of the mode of action, ERr 731® was administered orally by capsule to 3 male and 3 female dogs (pure-bred beagles, weight 6-9 kg, age 6-8 months) in a dose of 100 mg of ERr 731®/kg of body weight. After various times, blood was taken from the animals and blood plasma was obtained. The plasma was investigated for ERr 731® ingredients and metabolites. It was surprisingly possible to detect both in male and in female animals significant amounts of the metabolite piceatannol and small amounts of the metabolite resveratrol. Maximum plasma levels of these metabolites were reached after about 24 h. The plasma levels of piceatannol were distinctly higher than those of resveratrol. The results of the test at the 24 h timepoint are depicted in FIG. 2b.

The results of the tests described above surprisingly demonstrate that the main ingredients of ERr 731® are absorbed as glycosides in the body after oral administration and are detectable as such in the bloodstream in a dose-dependent manner, and thus are systemically bioavailable, whereas their direct aglycones rhapontigenin and deoxyrhapontigenin were undetectable. It was additionally possible to show that at high dosage the corresponding metabolites resveratrol and, in particular, piceatannol are also formed in the body.

### Test Example 2

### Effect of ERr 731® on IL-6

In vivo investigations on IL-6 levels in the blood took place during a 15-month observation study involving 82 patients with menopausal symptoms. These patients took one tablet of ERr 731® (Phytoestrol® N; dosage=4 mg of ERr 731®) once a day. Blood was taken before intake (IC) and after 3 months in each case, and IL-6 was detected in the serum by means of a specific ELISA (Pharmingen BD, Heidelberg).

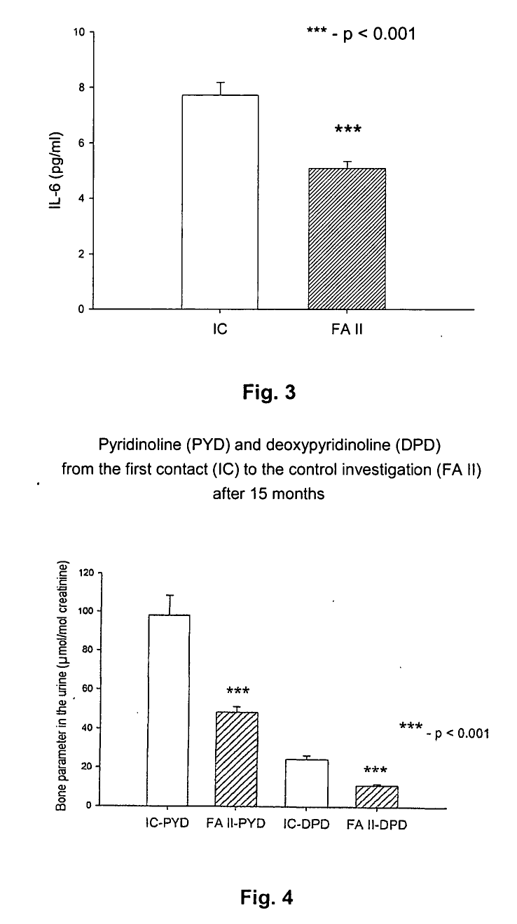

It was surprisingly found for the first time in these patients, after treatment for 15 months (FA II), that with ERr 731® the IL-6 levels were significantly reduced compared with the levels before the first intake (IC). This surprising finding is depicted in appended FIG. 3.

### Test Example 3

### Effect of ERr 731® on Markers of Bone Resorption

It was further possible to find in the patients investigated in test example 2, by means of enzyme immunoassays (Opsys Thermosystems, USA), a marked reduction in the markers of bone resorption pyridinoline (PYD) and deoxypyridinoline (DPD) in the urine. These surprising findings are depicted in appended FIG. 4.

In addition, no increase in further markers of bone resorption, such as, for example, ICTP-I and CTX, as important osteoporosis indicators, and no significant reduction in markers of bone formation, such as serum procollagen I carboxy-terminal propeptide (PICP) and serum procollagen I N-terminal propeptide (PINP) to be observed (experimental results not shown).

Overall, these are clear and surprising indications of an antiosteoporotic effect of ERr 731® in vivo.

### Test Example 4

### Effects of ERr 731® on the Cytokine-Stimulated Release of IL-6 in a Human Tumor Cell Model for Inflammatory Disorders

The human tumor cell line A549 (lung carcinoma cells) was used for the tests. These cells represent a model system for IL-6-producing cells in inflammatory disorders (Billich et al., Basal and induced sphingosine kinase 1 activity in A549 carcinoma cells: function in cell survival and IL-1β and TNF-α induced production of inflammatory mediators. Cell Signal 2005; 17: 1203-1217). A549 cells are human lung carcinoma cells (58-year old male patient, 1972) which have the ability to form tumors in suitable mouse models. A549 cells grow adherently, have a generation time of about 30 h and are cultured in FCS-containing (10%) DMEM cell culture medium. Stimulation was carried out with a combination of the following recombinant human cytokines:

IL-1β (50 ng/ml)
TNFα (50 ng/ml).

For the stimulation, confluent A549 cells (in 6-well plates) in DMEM stimulation medium (without phenol red, serum-free, in 0.01% fatty acid-free BSA) were activated with IL-1β/TNFα+/−ERr 731®.

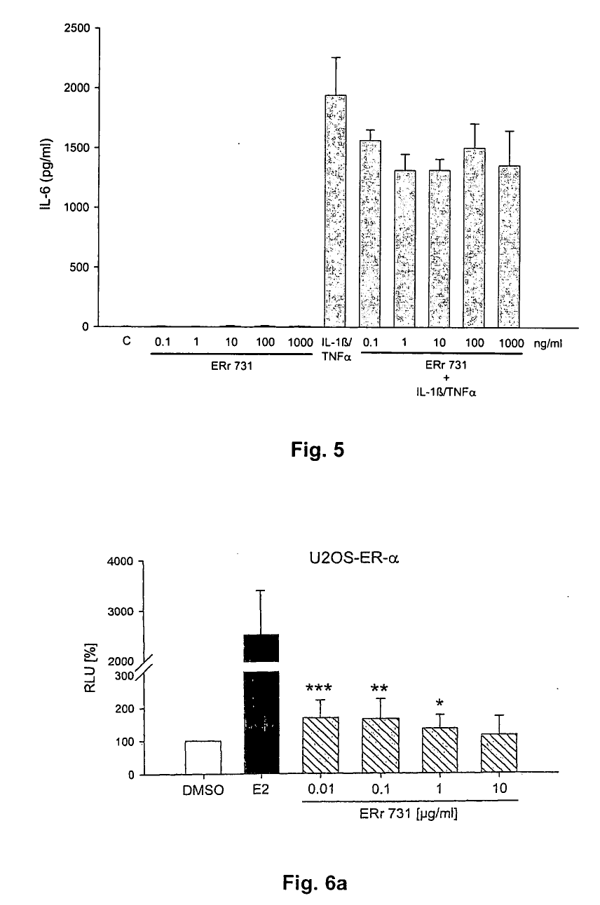

The respective extract concentrations (stock: 10 mg/ml in DMSO; tested concentrations: 0.1 ng/ml to 10 μg/ml) are evident from FIG. 5. During the stimulation, the DMSO concentration resulting from the highest final concentration of ERr 731® in the respective test series was generated in all culture mixtures (0.1% DMSO with 10 μg/ml, 0.01% DMSO with 1 μg/ml).

After incubation for 24 hours, the culture supernatants were removed by centrifugation and the relevant IL-6 concentrations in the cell-free supernatants (three dishes for each condition) were measured by duplicate determination using a specific ELISA for human IL-6.

The IL-1β/TNFα combination led in all test series to a robust induction of IL-6. The stimulatability of the A549 cells by IL-1β/TNFα varied on individual test days between 1000 pg/ml and 5000 pg/ml. The effects of ERr 731® showed no correlation to the strength of the initial stimulation in the test series.

A representative test result is depicted in appended FIG. 5:

It is seen that the inhibition of IL-6 release is about 28% with all ERr 731® concentrations. It was thus surprisingly possible for the first time to show that ERr 731®, i.e. its ingredients rhaponticin and rhapontigenin, bring about a partial reduction in IL-6 release from A549 cells. The observed effect on IL-6 production is surprising because it has previously been assumed that glycosidic hydroxystilbenes like those present in the active ingredient combination of the invention too have no effect on the mediator production in these cells (Donelly et al. Anti-inflammatory effects of resveratrol in lung epithelial cells: molecular mechanisms. Am J Physiol Lung Cell Mol Physiol 2004; 287: L774-L783).

### Test Example 5

### Activation of ER-α by ERr 731® in Various Cell Systems

### a) Osteosarcoma Cells

U2OS cells are human, epithelially organized osteosarcoma cells originally isolated in 1964 from the tibia of a 15-year old girl by J. Ponten and E. Saksela (Ponten and Saksela 1967: Two established in vitro cell lines from human mesenchymal tumours, Int. J. Cancer 2: 434-447).

A suitable medium for the cells is DMEM/F12 (Dulbecco's modified Eagle's Medium), to which 10% fetal calf serum was added for culturing the cells, and 5% fetal calf serum was added for the actual experiment. The latter was previously made steroid-free (DCC medium) using dextran-coated activated carbon (dextran-coated charcoal; DCC). Incubation took place at 37° C. with 5% v/v CO₂. In order to ensure comparability between the individual experiments, the passages of the cells used for the experiments never exceeded 20 after thawing from liquid nitrogen.

Besides native U2OS cells, use was made of U2OS-ERα cells (Schering A G, Berlin) which were stably transfected with ERα (pSG5 vector, Stratagene) and merely required transfection with the reporter gene (ERE)2-tk-Luc (pGL3 basic vector, Promega). The cells were transfected using the transfection reagent DOTAP (N-[1-(2,3-dioleoyloxy)]-N,N,N-trimethylammonium propane methylsulfate, Carl Roth GmbH & Co. KG), the ratio of DOTAP to total amount of DNA always being 3 μl of DOTAP to 1 μg of DNA.

Because the transfection efficiency of the cells differed it was necessary to use different mixtures for the U2OS cells and the U2OS-ERα cells. The optimized amounts of DNA and DOTAP employed are summarized in table 2.

TABLE 2
Parameters for transfection of U2OS and U2OS-ERα cells

| | U2OS | U2OS-ERα |
|---|---|---|
| DOTAP | 3 μl/1 μg total DNA | 3 μl/1 μg total DNA |
| pSG5-hERα | 100 ng | — |
| (ERE)2-tk-Luc | 600 ng | 100 ng |

After the cells were treated with the test or control substances for 24 h it was possible to measure the luciferase activity as reporter gene using a kit from Promega, as stated by the manufacturer. The protein content was determined using the BCA® kit (Sigma).

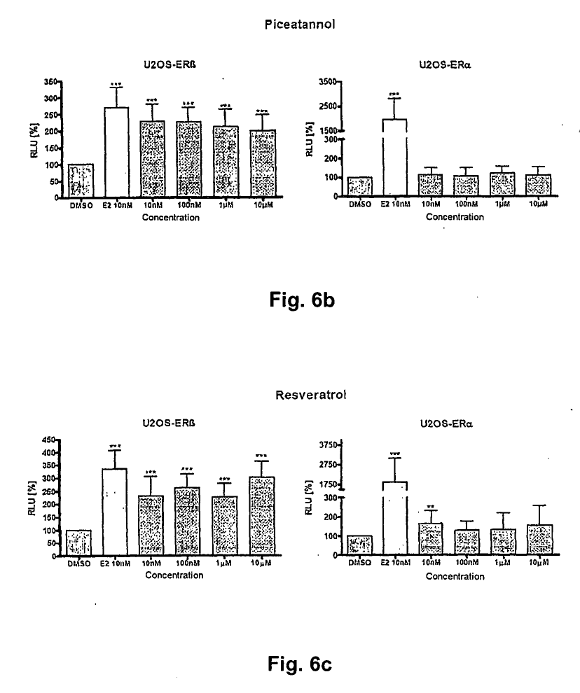

A weak, but significant activation of ER-α was measurable in the osteosarcoma cell line U2OS (bone cancer cells) stably transfected with ER-α even in the lowest concentrations of ERr 731®. The experimental results are summarized in appended FIG. 6a.

By contrast, the individual substances resveratrol and piceatannol showed no significant effects on ER-α activation in the U2OS osteosarcoma cells (FIG. 6 b, c).

### b) Recombinant Yeast Cells

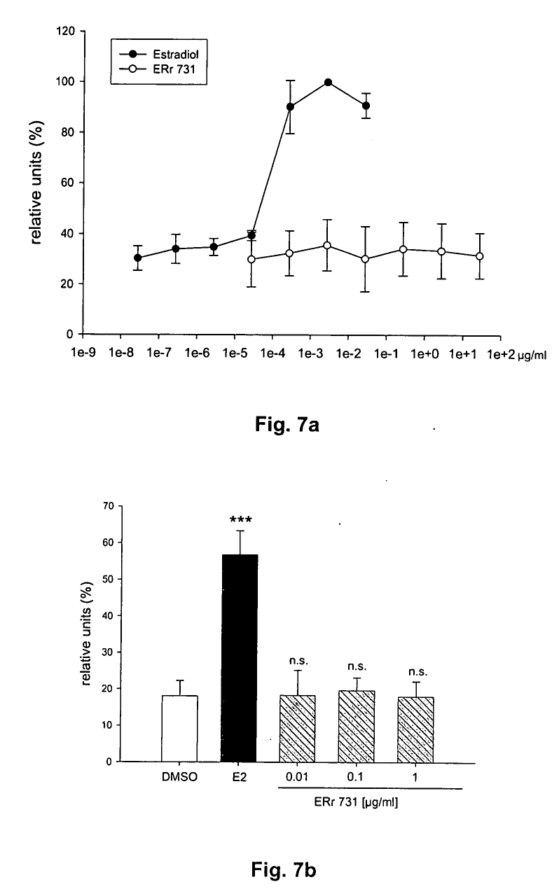

In a further series of experiments, a recombinant yeast screen was used (cf. E. Routledge and J. P. Sumpter, Östrogenic activity of surfactants and some of their degradation products assessed using a recombinant yeast screen, Envirom. Tox. Chem. 1996). Saccharomyces cerevisiae cells were stably transfected with human ERα both with a reporter gene consisting of the respective responsive promoter element fused to the LacZ gene which codes for β-galactosidase. Estrogen treatment (with estrogen or with a substrate having an estrogen-like effect) of the cells activates, mediated by the estrogen receptor, β-galactosidase, leading to a red coloration of the yeast cells, which can be measured at 565 nm. The test results are summarized in FIG. 7a.

### c) Ishikawa Cells

In a third series of experiments, the data of the estrogenicity measurement were verified by determining the induction of alkaline phosphatase in Ishikawa cells (human endometrial adenocarcinoma cells) which had been transfected with an ERα-containing reporter gene construct. The activity of alkaline phosphatase, which is assessed using the chromogenic substrate 4-nitrophenyl phosphate, represents an ERα-mediated response.

The test is based on the description by Holinka C F, Hata H, Kuramoto H, Gurpide E (1986) Effects of steroid hormones and antisteroids on alkaline phosphatase activity in human endometrial cancer cells (Ishikawa Line). Cancer Res. 46: 2771-2774, and modifications described in Wober J, Weiβwange I, Vollmer G (2002) Stimulation of alkaline phosphatase activity in Ishikawa cells induced by various phytoestrogens and synthetic estrogens. J. Steroid Biochem. Mol. Biol. 83:227-233.

Table 3 presents the concentrations for the positive control (estradiol) and the test substances used in the assay.

TABLE 3

| Test substance | Concentration (M)¹ |
|---|---|
| Estradiol | 10⁻⁶ |
| Resveratrol | 10⁻⁸-10⁻⁵ |
| trans-Rhapontigenin | 10⁻⁸-10⁻⁵ |
| Deoxyrhapontigenin | 10⁻⁸-10⁻⁵ |
| Piceatannol | 10⁻⁷-10⁻⁵ |
| cis-Rhapontigenin | 10⁻⁸-10⁻⁵ |
| Extract ERr 731^{®} | 0.00001-10 |

¹Exception: The concentration for the extract ERr 731 ® is indicated in μg/ml

The results of this test are depicted in FIG. 7b for ERr 731®.

### d) Summary

All tests are conclusive because all the positive and negative controls show their predicted effects (cf. FIGS. 6, 7a and 7b).

Since ER-α is essential for protection from osteoporosis, the positive effect on bone formation can be explained by a weak but constant activation of this receptor by ERr 731 (FIG. 6). The finding is surprising because ER-α was not activated by ERr 731® in all the other test systems (Ishikawa cells=human endometrial carcinoma line, yeast cells) which were transfected with ER-α (FIG. 7a,b). The active ingredient combination ERr 731® is thus a tissue-specific ER-α agonist.

Activation of ER-α in bone is osteoprotective but procarcinogenic in endometrial cells. It is therefore a decisive point that ER-α is not activated in endometrial cells, but is in bone cells, by ERr 731®. Thus, raloxifene which activates ER-α exclusively in bone but not in uterus, ovaries, endometrium (Nalbandian et al. The Selective Estrogen Receptor Modulators, Tamoxifen and Raloxifene, Impair Dendritic Cell Differentiation and Activation, J. Immunol. 2005; 175: 2666-2675) is now employed for the treatment of osteoporosis.

It has thus been shown for the first time that the active ingredient combination ERr 731® or an active ingredient combination of rhaponticin and deoxyrhaponticin can prevent osteoporosis.

1. A method of preventing and/or treating osteoporosis comprising administering to a subject a hydroxystilbene-containing active ingredient combination, wherein the active ingredient combination substantially comprises, in a ratio of about 10:1 to 1:5 by weight, rhaponticin and deoxyrhaponticin or stereoisomeric forms thereof, in each case in the form of their salts or in the phenol form, or ester or ether derivatives thereof.

2. The method as claimed in claim 1, where the active ingredient combination counteracts a pathological increase in the IL-6 serum level and/or shows an osteoprotective effect through a tissue-specific ERα activation.

3. The method as claimed in claim 1, wherein the active ingredient combination or the constituents thereof is (are) completely or partially chemically synthesized or can be isolated from plants.

4. The method as claimed in claim 1, wherein the active ingredient combination or the constituents thereof can be obtained completely or partially from plants of the genus Rheum sp., Astragalus spp., Cassia spp. or Picea sp.

5. The method as claimed in claim 1, wherein the active ingredient combination or the constituents thereof can be obtained completely or partially from roots and/or other plant parts of Rheum rhaponticum.

6. The method as claimed in claim 1, wherein the active ingredient combination substantially comprises rhaponticin and deoxyrhaponticin.

7. The method as claimed in claim 1, wherein, where the active ingredient combination comprises rhaponticin, deoxyrhaponticin, and rhapontigenin and/or deoxyrhapontigenin.

8. The method as claimed in claim 1, wherein the active ingredient combination is substantially free of aglycone derivatives of rhaponticin and deoxyrhaponticin.

9. The method as claimed in claim 8, wherein the active ingredient combination is substantially free of resveratrol and piceatannol.

10. The method as claimed in claim 1, wherein the active ingredient combination substantially consists of approximately:
60-70% by weight rhaponticin;
30-40% by weight deoxyrhaponticin;
0-2% by weight trans-rhapontigenin; and
0-2% by weight deoxyrhapontigenin.

11. The method as claimed in claim 1, wherein the active ingredient combination is combined with at least one further active ingredient which is suitable for the prevention and/or treatment of osteoporosis and differs from the compounds as defined in claim 1.

12. The method as claimed in claim 1, wherein the composition is selected from chemically synthesized, biotechnological produced, herbal and homeopathic medicaments, medicinal plant preparations, dietary supplements and dietetic food products.

13. A method for preventing and/or treating osteoporosis comprising administering to a subject a hydroxystilbene-containing active ingredient combination, wherein the active ingredient combination substantially comprises, in a ratio of about 10:1 to 1:5 by weight, rhaponticin and deoxyrhaponticin or stereoisomeric forms thereof, in each case in the form of their salts or in the phenol form, or ester or ether derivatives thereof and thereby reducing the IL-6 serum level and/or by tissue-specific activation of ERα.

14. The method of claim 13, wherein the tissue-specific activation of ERα is bone-specific activation.
